(19) United States
(12) Patent Application Publication
(10) Pub. No.: US 2025/0340502 A1
(43) Pub. Date: Nov. 6, 2025

(54) PURIFICATION OF AN ETHYLENICALLY UNSATURATED ALCOHOL STREAM, PREPARATION OF AN ETHYLENICALLY UNSATURATED ALDEHYDE, IN PARTICULAR PRENAL, AND COMPOUNDS DERIVED THEREFROM

(71) Applicant: BASF SE, Ludwigshafen am Rhein (DE)

(72) Inventors: Nicolas VAUTRAVERS, Ludwigshafen am Rhein (DE); Gabriele IFFLAND, Ludwigshafen am Rhein (DE); Maria Daniela GAMERO, Ludwigshafen am Rhein (DE); Jessica Nadine HAMANN, Ludwigshafen am Rhein (DE); Andreas Joerg UFER, Ludwigshafen am Rhein (DE); Joaquim Henrique TELES, Ludwigshafen am Rhein (DE)

(21) Appl. No.: 18/867,033

(22) PCT Filed: May 19, 2023
(86) PCT No.: PCT/EP2023/063508

§ 371 Date: Nov. 19, 2024

(30) Foreign Application Priority Data
May 20, 2022 (EP) 22174556.5

(51) Int. Cl. C07C 29/88

(52) U.S. Cl. CPC C07C 29/88; C07C 2523/42; C07C 2523/44; C07C 2523/52

(57) ABSTRACT

Organically bound nitrogen is removed from an ethylenically unsaturated alcohol stream by contacting the alcohol stream with a weakly acidic solid adsorbent. Trace amounts of organically bound nitrogen tend to poison the oxidation catalyst in subsequent oxidation processes using the ethylenically unsaturated alcohol stream.

The invention relates to a process for purifying an ethylenically unsaturated alcohol stream, and more particularly for removing organically bound nitrogen from an ethylenically unsaturated alcohol stream, and to a process for the preparation of an ethylenically unsaturated aldehyde from an ethylenically unsaturated alcohol, in particular prenal from prenol, in the presence of an oxidant and a catalytically active metal catalyst. The invention further relates to processes for the preparation of 3,7-dimethyl-octa-2,6-dienal (citral), menthol and linalool derived.

Ethylenically unsaturated aldehydes such as prenal (3-methyl-2-buten-1-al) are important chemical intermediates, e.g. for the preparation of terpene-based fragrances, such as citral, and for the preparation of vitamins, such as vitamin E. Therefore, such ethylenically unsaturated aldehydes are of great technical and economic importance. The literature provides various examples for the preparation of ethylenically unsaturated aldehydes.

The WO 2009/106621 A1 describes a process for producing olefinically unsaturated carbonyl compounds, e.g. prenol, by oxidative dehydrogenation of e.g. prenol and/or isoprenol. The reaction is carried out at temperatures in the range of from 50 to 240° C. in an oxygen-containing atmosphere on a supported gold-containing catalyst. For example, p-xylene may act as a solvent.

The WO 2018/002040 A1 describes a process for the preparation of α,β-unsaturated aldehydes by oxidation of alcohols using oxygen or air as oxidant in the presence of a catalyst comprising platinum on a support. The reaction is carried out in the presence of a liquid phase which contains at least 25 wt.-% of water, based on the total weight of the liquid phase.

The WO 2018/172110 A1 describes a process for the preparation of α,β-unsaturated aldehydes, e.g. prenal, by oxidation of alcohols, e.g. prenol, in the presence of a liquid phase. Said liquid phase contains 0.1 to less than 25 wt.-% of water and at least 25 wt.-% of alcohol(s), e.g. prenol, and α,β-unsaturated aldehyde(s), e.g. prenal. Oxygen and/or hydrogen peroxide may be used as oxidant. Preferably, the oxidation is carried out in the presence of a catalyst comprising a catalytically active metal, selected from platinum, palladium and gold, on a support.

The WO 2019/121012 A1 describes a process for the preparation of α,β-unsaturated aldehydes, e.g. prenal, by oxidation of alcohols, e.g. prenol, in the presence of a liquid phase comprising at least 25 wt.-% of water. Oxygen is used as oxidant. The oxidation is carried out in the presence of a catalyst comprising a catalytically active metal on a support, wherein the catalytically active metal is located mainly in the outer shell of the catalyst. Preferably, the catalytically active metal is selected from platinum, palladium and gold.

The WO 2019/121011 A1 describes a process for the preparation of prenal (3-methylbut-2-en-1-al) from dimethylvinyl carbinol (2-methylbut-3-en-2-ol) which may optionally further contain prenol. It is assumed that 2-methyl-3-buten-2-ol is isomerized to 3-methyl-2-buten-1-ol, which is subsequently oxidized to 3-methyl-2-buten-1-al. The process is carried out in the presence of an oxidant and a catalyst. The catalyst comprises a catalytically active metal which preferably is on a support, wherein the catalytically active material is preferably selected from platinum, palladium and gold. Furthermore, the process is carried out at a pH of less than 7, wherein the pH may be adjusted by addition of e.g. an acid or a strongly acidic cation exchanger. Suitably, the process is carried out in the presence of a liquid phase comprising at least 25 wt.-% of water.

These catalysts having a noble metal deposited on a support exhibit good alcohol conversion and excellent selectivity, and may exhibit, depending upon the exact nature of the catalyst, long lifetime. Over time, however, these catalysts may lose some activity, and occasionally, may become sufficiently deactivated so as to render the catalyst impractical to use. At this stage of partial or full deactivation, the catalyst must be regenerated or replaced.

The present invention is based on the insight that trace amounts of organically bound nitrogen, for example, nitrogen in the form of amines, tend to poison the oxidation catalyst. Isoprenol is produced by the chemical condensation of isobutene and formaldehyde, leading to isoprenol further isomerized to prenol. The reaction of isobutene and formaldehyde may be carried out in the presence of a catalyst such as an amine base, e.g. hexamethylenetetramine (urotropin), as e.g. described in U.S. Pat. No. 3,574,773. The amine base also intercepts the formic acid formed by disproportionation of the formaldehyde. As a result, the prenol and/or isoprenol streams obtained may, for example, contain organically bound nitrogen impurities in an amount of several ppm.

However, the reliable purification of ethylenically unsaturated alcohols from trace amounts of organically bound nitrogen on an industrial scale is not a trivial task. Generally, such ethylenically unsaturated alcohols are reactive compounds. Said reactivity may lead to difficulties, e.g. when performing purification steps for removing impurities from such ethylenically unsaturated alcohols. For example, upon treatment with a solid adsorbent, isoprene is suspected to form a tertiary carbocation that can give rise to undesired side reactions. In addition, the nitrogen compounds, which are only present in low concentrations, compete with the abundant alcohol for absorption sites.

The DE 199 10 504 A1 describes a process for reducing the amine content, e.g. mono-methylamine, of amine-contaminated N-substituted lactams, e.g. N-methyl-2-pyrroli-done, by treating the contaminated N-substituted lactams with an acidic macroporous cation exchanger. At the same time, metal cations contained as impurities in the N-substituted lactams may be depleted.

Thus, there remains a need for feasible processes for removing organically bound nitrogen from feed streams such as ethylenically unsaturated alcohol streams. Furthermore, there remains a need for improved processes for the preparation of ethylenically unsaturated aldehydes.

The object of the present invention is solved by a process for removing organically bound nitrogen from an ethylenically unsaturated alcohol stream, by contacting the alcohol stream with a weakly acidic solid adsorbent.

By the process of the present invention, an ethylenically unsaturated alcohol stream is depleted of organically bound nitrogen. Thus, the invention allows for overcoming the above problems in subsequent processes using the ethylenically unsaturated alcohol stream. For example, such subsequent processes include oxidations of said ethylenically unsaturated alcohols.

Generally, the alcohol stream is contacted with the weakly acidic solid adsorbent in the substantial absence of an oxidant. Generally, the alcohol stream is contacted with the weakly acidic solid adsorbent in the absence of a catalytically active metal catalyst.

Herein, the term “organically bound nitrogen” is intended to denote any compound containing at least one nitrogen atom directly bound to one or more carbon atoms. For example, such compounds containing at least one nitrogen atom may be selected from amines, such as ethylamine, trimethylamine, aniline, pyridine or piperidine. An amine particularly significant in practice is hexamethylenetetramine (urotropin). Ethylenically unsaturated alcohol streams, for example prenol or isoprenol streams, that are subjected to the method of the invention may comprise about 5 to 30 ppm of organically bound nitrogen.

The “ethylenically unsaturated alcohol stream” of the invention comprises an alcohol having a carbon-carbon double bond. For example, the ethylenically unsaturated alcohol stream may be an α,β-unsaturated alcohol or a β,γ-unsaturated alcohol.

Examples of suitable ethylenically unsaturated alcohols include 3-butene-1-ol, 3-pentene-1-ol, 3-methylbut-3-en-1-ol, 3-methylbut-2-en-1-ol, 3-hexene-1-ol, 3-methylpent-3-en-1-ol, 3-ethylbut-3-en-1-ol, 2-methylhex-1-en-5-ol, 2-methylhex-1-en-4-ol, 2-phenylbut-1-en-4-ol, 4-methylpent-3-en-1-ol and 2-cyclohexylbut-1-en-4-ol, and mixtures thereof.

In a preferred embodiment, the ethylenically unsaturated alcohol is selected from 3-methylbut-2-en-1-ol (prenol), 3-methylbut-3-en-1-ol (isoprenol), and mixtures thereof, and is preferably 3-methylbut-2-en-1-ol (prenol).

According to the invention, the alcohol stream is contacted with a weakly acidic solid adsorbent. Such solid adsorbents in the context of the present invention have been found to be capable of adsorbing organically bound nitrogen in the presence of abundant alcohol while not interfering with the reactive carbon-carbon double bond.

The weakly acidic adsorbent may include an adsorbent material having sufficient acidity to adsorb the organically bound nitrogen from the stream.

In an embodiment, the solid adsorbent is a crosslinked resin having phosphonic functional groups.

Preferably, the resin polymer is a vinyl aromatic copolymer, preferably crosslinked polystyrene and more preferably a polystyrene divinylbenzene copolymer. Other polymers having a phosphonic functional group may also be used.

Preferably, the crosslinked resin having phosphonic functional groups is of the macroporous type.

A preferred solid adsorbent is Purolite S956.

The resin is typically used in bead form and loaded into a column. The stream is passed through the column, contacting the resin beads. During contact, the organically bound nitrogen in the stream reacts with the functional group and an exchange occurs where a proton is transferred to the nitrogen and an ionic bond is formed to the anionic site of the resin. Contact is maintained until a threshold level is reached i.e. the breakthrough concentration. At this breakthrough point, the process reaches an equilibrium where additional organically bound nitrogen cannot be removed effectively. The flow is halted and the column is backwashed with water, preferably deionized or softened water. By flowing in reverse, the resin is fluidized and solids captured by the beads are loosened and removed.

In another embodiment, the solid adsorbent is a silica-alumina hydrate. Numerous silica-alumina catalyst compositions and processes for their preparation are described in the patent literature, see, e.g., U.S. Pat. No. 4,499,197.

Preferably, the alumina content of the silica-alumina hydrate is from about 10 to about 15 90 wt.-% of Al₂O₃. The preferred range of alumina content is from about 30 to about 70 wt.-% of Al₂O₃.

The introduction of silicon dioxide into aluminum oxide leads to the introduction of acidic centers. The number of acidic centers can be controlled by the amount of introduced silicon dioxide. The number of acidic centers increases with the amount of introduced silicon dioxide up to a maximum number of acidic centers, and decreases again with a further increasing amount of silicon dioxide after having reached the maximum number of acidic centers.

Examples of commercially available silica-alumina hydrates are Siral® available from Sasol Germany Gmbh, Hamburg, Germany. Siral® is based on orthorhombic aluminum oxide hydroxide (boehmite; AlOOH) and doped with SiO₂. Various Siral® grades having different ratios of Al₂O₃ to SiO₂ are available: Siral 1 (Al₂O₃/SiO₂=99/1), Siral 5 (Al₂O₃/SiO₂=95/5), Siral 10 (Al₂O₃/SiO₂=90/10), Siral 20 (Al₂O₃/SiO₂=80/20), Siral 28M (Al₂O₃/SiO₂=72/28), Siral 30 (Al₂O₃/SiO₂=70/30), Siral 40 (Al₂O₃/SiO₂=60/40). Siral 40 is especially preferred.

In a suitable determination method, the solid adsorbent is characterized by temperature programmed desorption of ammonia (TPAD) carried out on an apparatus constructed from Raczek analyzing technique GmbH, Hannover (Germany). For this purpose, the samples are conditioned at a temperature of 400° C. in helium flow. Afterwards, a mixture of 10% NH₃/He is passed over the sample at 70° C. The physisorbed ammonia is removed by flushing with helium at 120° C. for 2 h. The chemisorbed ammonia is removed by passing helium over the sample which was heated up to 400° C. with a linear heating rate of 15° C./min. The integration values of the peaks in the amount of ammonia that desorbs from the solid adsorbent is reported as amount of acidic centers.

In an embodiment, the ethylenically unsaturated alcohol stream is passed over a bed of the weakly acidic solid adsorbent.

Suitably, said step of “passing over a bed” denotes that a layer (“bed”) of the weakly acidic solid adsorbent is provided in a customary reaction vessel known to the skilled person which may preferably be equipped with a stirring device, e.g. in a stirred-tank reactor. The ethylenically unsaturated alcohol stream is then introduced into the reaction vessel and guided through the same in a manner that it gets into contact with the weakly acidic solid adsorbent.

Alternatively, the weakly acidic solid adsorbent may be provided in a reaction tube, e.g. of a tubular reactor and the ethylenically unsaturated alcohol stream then continuously flows through said reaction tube(s) while getting into contact with the weakly acidic solid adsorbent.

In an embodiment, the ethylenically unsaturated alcohol stream comprises, after contacting the alcohol stream with a weakly acidic solid adsorbent, less than 2 ppm of organically bound nitrogen. Herein, “ppm” denotes wt.-ppm of compounds incorporating organically bound nitrogen, relative to the total weight of the ethylenically unsaturated alcohol stream.

Suitably, the content of organically bound nitrogen in the ethylenically unsaturated alcohol stream may be determined by Kjeldahl analysis. Alternatively, an oxidative combustion method with a chemiluminescence detector according to DIN 51444 may be used.

The invention further relates to a process for the preparation of an ethylenically unsaturated aldehyde from an ethylenically unsaturated alcohol in the presence of an oxidant and a catalytically active metal catalyst, wherein prior to contacting with the catalytically active metal catalyst, the ethylenically unsaturated alcohol stream is treated by a process as described above.

Herein, said process for the preparation of an ethylenically unsaturated aldehyde is referred to as “alcohol dehydrogenation process”.

In other words, the ethylenically unsaturated alcohol stream is treated by the process for removing organically bound nitrogen from an ethylenically unsaturated alcohol stream, by contacting the alcohol stream with a weakly acidic solid adsorbent. After said treatment, the treated ethylenically unsaturated alcohol stream is then reacted in the presence of an oxidant and a catalytically active metal catalyst, as will be described in the following.

This procedure allows for catalyst activity and catalyst lifetime to be improved. Furthermore, conversion and selectivity of the subsequent oxidation reaction is enhanced.

The alcohol dehydrogenation process is carried out in the presence of an oxidant. In a preferred embodiment, the oxidant is selected from oxygen and hydrogen peroxide.

The oxidant may be a gas mixture containing oxygen in a content in the range of 2 to 50% by volume, preferably of 3 to 40% by volume, more preferably 7 to 18% by volume. Apart from oxygen, the gas mixture may further contain diluent gases. Suitably, diluent gases are inert gases such as nitrogen, argon, carbon dioxide etc. For example, the oxidant may be air as a readily available oxidation medium.

Preferably, oxygen is used undiluted.

The alcohol dehydrogenation process is carried out in the presence of a catalytically active metal catalyst.

Suitably, the catalytically active metal may be selected from metals of groups 8, 9, 10 and 11 of the periodic table of elements according to IUPAC. The elements of groups 8, 9, 10 and 11 comprise iron, cobalt, nickel, copper, ruthenium, rhodium, palladium, silver, osmium, iridium, platinum, and gold.

In a preferred embodiment, the catalytically active metal is selected from platinum, palladium and gold. Platinum is especially preferred.

The catalytically active metal can be used in any form, e.g. unsupported or on a support.

The catalytically active metal can be used in an unsupported form, for example as a powder, a mesh, a sponge, a foam or a net.

In a preferred embodiment, the catalytically active metal is deposited on a support which is preferably selected from carbonaceous materials and oxidic materials.

For example, the support may comprise aluminum oxide, silicon dioxide, magnesium oxide, or hydrotalcite.

In principle, suitable support materials are basic, acidic or else amphoteric support materials. Basic materials have been found to be particularly suitable. Aluminum oxides, basic aluminosilicates or hydrotalcites, preferably aluminum oxides and hydrotalcites, have been found to be advantageous in some cases. Support materials based on carbon, for example various kinds of charcoal, are also suitable.

In a preferred embodiment, the catalytically active metal is platinum which is on a support, wherein the support is selected from carbonaceous materials and oxidic materials, and wherein the oxide is selected from oxides of Al, Ce, Zr, Ti, V, Cr, Zn and Mg, preferably Al, Ce, Zr and Ti.

In an especially preferred embodiment, the catalyst is selected from platinum on carbon (Pt/C) and platinum on aluminum oxide (Pt/Al₂O₃).

In the case that the catalytically active metal is deposited on a support, the total weight of the catalyst herein shall be defined as the sum of the weight of the catalytically active metal and the weight of the support.

The content of the catalytically active metal in the catalyst is not subject to any particular restriction per se and may be in the range of from 0.1 to 20 wt.-%, preferably 0.1 to 15 wt.-%, more preferably 0.5 to 10 wt.-%, based on the total weight of the catalyst.

Optionally, the catalyst may comprise one or more promotors which enhance the activity of the catalytically active metal. Examples for such promotors are bismuth, antimony, lead, cadmium, tin or tellurium. For example, the promotors may be present on or in the support or can be added separately to the process.

In an embodiment, the process is carried out at a temperature in the range of from 1 to 250° C., preferably 5 to 150° C., more preferably 20 to 100° C., most preferably, 25° C. to 80° C., in particular 30 to 70° C., in particular 35 to 50° C.

In an embodiment, the reaction is performed in the liquid phase, which contains at least 25 wt.-%, preferably at least 30 wt.-%, more preferably at least 40 wt.-%, in particular at least 50 wt.-% of water, determined at a temperature of 20° C. and a pressure of 1 bara and based on the total weight of the liquid phase.

Optionally, the liquid phase may further comprise one or more solvent(s). Herein, the term “solvent” denotes any component other than reactant(s), product(s), oxidant(s), or water, which is liquid at a temperature of 20° C. and a pressure of 1 bara.

The liquid phase may comprise less than 70 wt.-%, preferably less than 60 wt.-%, more preferably less than 50 wt.-%, most preferably less than 40 wt.-%, in particular less than 30 wt.-%, in particular less than 20 wt.-%, in particular less than 10 wt.-%, determined at a temperature of 20° C. and a pressure of 1 bara and based on the total weight of the liquid phase.

Preferably, the solvent is an aprotic organic solvent. Useful aprotic organic solvents are selected from
aliphatic hydrocarbons, such as hexane, heptane, octane, nonane, decane and petroleum ether;
aromatic hydrocarbons, such as benzene, toluene, xylenes and mesitylene;
aliphatic ethers, such as 1,2-dimethoxy-ethane (DME), diethylene glycol dimethyl ether (diglyme), diethyl ether, dipropyl ether, methyl isobutyl ether, tert-butyl methyl ether, tert-butyl ethyl ether, dimethoxymethane, diethoxymethane, dimethylene glycol dimethyl ether, dimethylene glycol diethyl ether, trimethylene glycol dimethyl ether, trimethylene glycol diethyl ether and tetramethylene glycol dimethyl ether;
cycloaliphatic hydrocarbons, such as cyclohexane and cycloheptane;
alicyclic C₃-C₆-ethers, such as tetrahydrofuran (THF), tetrahydropyran, 2-methyl-tetrahydrofuran, 3-methyltetrahydrofuran, 1,3-dioxolane, 1,4-dioxane and 1,3,5-tri-oxane;
short-chain ketones, such as acetone, ethyl methyl ketone and isobutyl methyl ketone;
C₃-C₆-esters, such as methyl acetate, ethyl acetate, methyl propionate, dimethyloxalate, methoxyacetic acid methyl ester, ethylene carbonate, propylene carbonate, ethylene glycol diacetate and diethylene glycol diacetate;
C₃-C₆-amides, such as dimethylformamide (DMF), dimethylacetamide and N-methyl-pyrrolidone (NMP);
sulfoxides, such as dimethyl sulfoxide (DMSO);
C₃-C₆-nitriles, such as acetonitrile and propionitrile; and
mixtures thereof.

Preferably, the solvent has a boiling point above 50° C., more preferably in the range of from 50 to 200° C., most preferably 65 to 180° C., in particular 80 to 160° C.

In an embodiment, the liquid phase contains 1 to 75 wt.-%, preferably 1 to 50 wt.-%, more preferably 2 to 45 wt.-%, most preferably 3 to 40 wt.-% of the ethylenically unsaturated alcohol, based on the total amount of the liquid phase.

By this measure it is ensured that a sufficient amount of starting material, i.e. ethylenically unsaturated alcohol, is present in the reaction mixture of the alcohol dehydrogenation process.

The alcohol dehydrogenation process may be carried out in reaction vessels customary for such reactions. For example, the alcohol dehydrogenation process may be carried out in a continuous, semi-batch or batch-wise mode. Suitable reaction vessels are known to the skilled person in the art.

The alcohol dehydrogenation process can be carried out under atmospheric pressure or under pressure. Preferably, the alcohol dehydrogenation process is however carried out under a pressure in the range of more than 1 to 15 bara, more preferably 1 to 10 bara.

In the event that oxygen is used as the oxidant, the alcohol dehydrogenation process may suitably be carried out at a partial pressure of oxygen in the range of from 0.1 to 15 bara, preferably 0.2 to 10 bara, more preferably 0.2 to 8 bara, most preferably 0.2 to 5 bara, in particular 1 to 3 bara, in particular 1 to 2.5 bara, in particular 1.2 to 2 bara.

The obtained crude product(s) from the alcohol dehydrogenation process may be subjected to conventional purification measures known to the skilled person in the art, including distillation or chromatography, or combinations thereof. Suitable distillation devices for the purification of the product(s) include, for example, distillation columns, such as tray columns optionally equipped with bubble cap trays, sieve plates, sieve trays, packages or filler materials, or spinning band columns, such as thin film evaporators, falling film evaporators, forced circulation evaporators, wiped-film (Sambay) evaporators, etc., and combinations thereof.

A variety of ethylenically unsaturated aldehydes can be produced by the invention. In one embodiment, the ethylenically unsaturated alcohol is 3-methylbut-2-en-1-ol (prenol) and the ethylenically unsaturated aldehyde produced is prenal (3-methyl-2-buten-1-al).

Prenol useful as a starting material for the invention may be obtained by reacting a formaldehyde source and isobutylene to obtain 3-methylbut-3-en-1-ol (isoprenol), and subjecting the obtained isoprenol to isomerization.

In one embodiment, the isoprenol is obtained by mixing and injecting a formaldehyde source and isobutylene into a reactor through at least one nozzle and reacting the formaldehyde source and isobutylene under supercritical conditions. In order to achieve supercritical conditions, formaldehyde and isobutylene are preferably reacted at a temperature of at least 220° C., for example in the range of 220 to 290° C., and an absolute pressure of at least 200 bara. The reaction of isobutene and formaldehyde may be carried out in the presence of a catalyst such as an amine base, e.g. hexamethylenetetramine (urotropin).

Formaldehyde may be provided as a liquid, for example as a solution of paraformaldehyde. Preferably, the formaldehyde source is an aqueous formaldehyde solution.

Further details regarding reacting a formaldehyde source and isobutylene to obtain isoprenol may be found in WO 2020/049111 A1.

Separation of the isoprenol from formaldehyde is not a trivial task. This difficulty arises from the fact that monomeric formaldehyde (as well as polymeric formaldehyde) forms both hydrates with water and hemiformals with isoprenol. The hydrates and hemiformals of varying formaldehyde polymerization degree have intermingling boiling points.

It has, however, been found that formaldehyde can be separated virtually completely from isoprenol via distillation at a temperature at which the hemiformal is cleaved to formaldehyde and isoprenol, so that the formaldehyde can be easily separated from the isoprenol.

Hence, crude isoprenol may be purified by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.

In particular, it has been found that the formaldehyde can be separated virtually completely from isoprenol and concentrated aqueous formaldehyde suitable for recycling into the isoprenol synthesis can be obtained in a distillation train involving a first distillation at a temperature at which the equilibrium is shifted towards the hemiformal of formaldehyde and isoprenol, so that essentially all formaldehyde remains in the bottoms of the distillation, and a second distillation at a temperature at which the hemiformal is cleaved to formaldehyde and isoprenol, so that the formaldehyde can be easily separated from the isoprenol.

In order to permit a first distillation at a temperature below the isoprenol-formaldehyde dissociation temperature and a second distillation at a temperature above the isoprenol-formaldehyde dissociation temperature, two low-boiler separation towers operated at different pressures are envisioned. Hence, at the relatively low pressure prevailing in the first low-boiler separation tower, a first distillate containing water and low-boilers essentially free of formaldehyde is obtained. At the relatively high pressure prevailing in the second low-boiler separation tower, a virtually all formaldehyde is separated from the isoprenol. This process thus allows for obtaining isoprenol essentially free of formaldehyde.

Hence in a more preferred embodiment, the purification process comprises
(i) directing the stream of crude isoprenol to a first low-boiler separation tower operated at a pressure of 1.5 bara or lower, to obtain a first bottoms stream containing isoprenol and formaldehyde, and a first distillate stream containing water and low-boilers;
(ii) directing the first bottoms stream to a second low-boiler separation tower operated at a pressure of 2 bara or higher, to obtain a second distillate stream containing aqueous formaldehyde, and a second bottoms stream containing isoprenol; and
(iii) directing the second bottoms stream to a finishing tower to obtain pure isoprenol as a distillate stream, and a bottoms stream containing high-boilers.

The second distillate stream constitutes concentrated aqueous formaldehyde fit for recycle into the isoprenol synthesis.

Suitably, the second low-boiler separation tower is operated at a pressure of 2.5 bara or higher, preferably 2.8 bara or higher, most preferably 2.9 bara or higher. The bottoms temperature of the second low-boiler separation tower is preferably in the range of 160 to 200° C., more preferably 170 to 185° C., most preferably 175 to 180° C. The temperature at the top of the second low-boiler separation tower is preferably in the range of 115 to 160° C., more preferably 125 to 145° C.

In a particularly preferred embodiment, the second low-boiler separation tower is operated at a pressure in the range of 2.9 to 3.5 bara, a bottoms temperature in the range of 175 to 180° C. and a temperature at the top in the range of 130 to 140° C.

Further information on the process for recovering isoprenol essentially free of formaldehyde may be found in WO 2022/189652 A1.

The obtained isoprenol is subjected to catalytic isomerization so as to obtain prenol.

Isomerization of isoprenol to 3-methyl-2-buten-1-ol (prenol) may be carried out over a supported noble metal, preferably in the presence of hydrogen. A preferred catalyst is a fixed bed catalyst containing palladium and selenium or tellurium or a mixture of selenium and tellurium supported on silicium dioxide. The isomerization is carried out at a temperature of 50 to 150° C. to produce a reaction mixture of prenol and isoprenol. The isoprenol can be recycled. Further details are provided in WO 2008/037693.

The prenal produced by the invention is a useful intermediate in the preparation of citral. Citral is a mixture of the isomeric compounds neral and geranial.

3,7-dimethyl-octa-2,6-dienal (citral) can be prepared by obtaining prenal by a process as described above, further comprising the steps of condensing the prenal with prenol to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

In particular, 3,7-dimethyl-octa-2,6-dienal (citral) can be prepared by a process comprising the steps of:
a) condensing the prenal with prenol in the presence of at least one catalyst in a reaction column while withdrawing an acetal fraction comprising the diprenyl acetal of prenal from the reaction column;
b) subjecting the acetal fraction in a cleaving column to cleaving conditions in the presence of at least one catalyst while withdrawing from the cleaving column a cleaving fraction containing at least one of prenyl (3-methyl-butadienyl) ether and 2,4,4-trimethyl-3-formyl-1,5-hexadiene, and optionally containing citral; and
c) reacting the cleaving fraction in a plug-flow type reactor to obtain citral.

The overall reaction sequence is illustrated by the reaction scheme below.

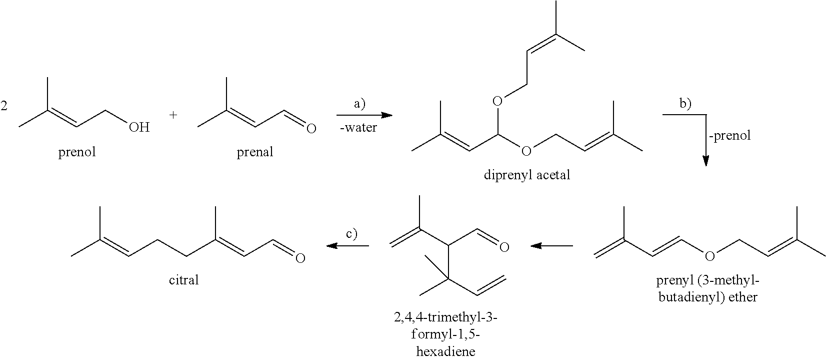

In step a), the unsaturated acetal 3-methyl-2-butenal-diprenyl acetal (herein referred to as “diprenyl acetal of prenal” or “diprenyl acetal”) is formed from prenol and prenal using a catalyst. For this purpose, prenal is reacted together with prenol in the presence of catalytic amounts of an acid and with separation of the water formed during the reaction in a reaction column. In step b), the resulting 3-methyl-2-butenal diprenyl acetal (diprenyl acetal) of step a) is cleaved in the presence of a catalyst in a cleaving column with elimination of 3-methyl-2-buten-1-ol (prenol) to give prenyl (3-methylbutadienyl) ether. Claisen rearrangement of the obtained prenyl (3-methylbutadienyl) ether yields 2,4,4-trimethyl-3-formyl-1,5-hexadiene which subsequently undergoes Cope rearrangement yielding 3,7-dimethyl-2,6-octadienal (citral).

Step a) is carried out in the presence of a catalyst, preferably an acid. In an embodiment, the catalyst in step a) is nitric acid.

Preferably, in step b), the acetal fraction is continuously subjected to cleaving conditions in a cleaving column. “Cleaving conditions” denotes reaction conditions selected such that the diprenyl acetal contained in the acetal fraction is cleaved to prenyl (3-methylbutadienyl) ether which may subsequently rearrange to 2,4,4-trimethyl-3-formyl-1,5-hexadiene and citral.

The acetal fraction comprises diprenyl acetal as a main constituent. The acetal fraction does not necessarily need to consist of pure diprenyl acetal, but may also comprise prenol, prenal and citral building blocks.

Step b) is carried out in the presence of a catalyst, preferably an acid catalyst. Suitable acid catalysts are selected from non-volatile protic acids such as sulfuric acid, p-toluenesulfonic acid and phosphoric acid.

Suitably, the continuous cleaving in the cleaving column of step b) may be carried out in the lower part or the sump of the distillation column acting as cleaving column. Preferably, the acetal fraction and/or the catalyst(s) are introduced into the lower part of the distillation column, into the sump of the distillation column or into the evaporator of the distillation column.

If desired, a high-boiling inert compound can be introduced into the sump of the cleaving column in order to ensure a minimum filling level of the sump and the evaporator. Suitable high-boiling inert compounds are selected from liquid compounds which are inert under the reaction conditions and have a higher boiling point than citral and diprenyl acetal. For example, the high-boiling inert compounds may be selected from hydrocarbons such as tetradecane, pentadecane, hexadecane, octadecane, eicosane; or ethers such as diethylene glycol dibutyl ether; white oils; kerosene oils; or mixtures thereof.

Suitably, the distillation conditions are selected such that the diprenyl acetal is predominantly retained in the lower part or the sump of the distillation column. During the cleaving reaction, a cleaving fraction is continuously withdrawn from the cleaving column, the cleaving fraction containing at least one of prenyl (3-methyl-butadienyl) ether and 2,4,4-trimethyl-3-formyl-1,5-hexadiene, and optionally containing citral. For the ease of reference, prenyl (3-methyl-butadienyl) ether, 2,4,4-trimethyl-3-formyl-1,5-hexadiene and citral are collectively referred to as “citral building blocks”. This is because the former are intermediates on the reaction route to citral and can be converted into citral in the subsequent step c).

Additionally, the prenol formed during the cleaving reaction in step b) is continuously removed from the reaction mixture, generally at the top of the cleaving column.

The cleaving fraction together with the formed prenol may be withdrawn at the top of the distillation column.

Alternatively and preferably, it is also possible to withdraw the cleaving fraction in liquid or vaporous form at a side draw of the distillation column.

In step c), the cleaving fraction is reacted in a plug-flow type reactor to obtain citral. To this end, the cleaving fraction is guided through the plug-flow type reactor at a suitable temperature for carrying out the rearrangement reaction(s) yielding citral. By employing a combination of a highly back-mixed cleaving column and a plug-flow reactor, it is possible to increase the selectivity and the yield of the cleaving reaction. All of the catalyst(s) required for the cleaving reaction is preferably introduced into the cleaving column in step b) and preferably, no catalyst is introduced into the plug-flow reactor.

In an embodiment, prenol eliminated in step b) is recycled to step a). This allows for improved yields to be achieved in the process of the invention.

In one aspect, the invention hence relates to an improved process for the preparation of citral (3,7-dimethyl-octa-2,6-dienal), comprising the steps of
A) reacting a formaldehyde source and isobutylene to obtain 3-methylbut-3-en-1-ol (isoprenol), and subjecting at least part of the obtained isoprenol to isomerization to obtain prenol;
B) preparing prenal (3-methylbut-2-en-1-al) from 3-methylbut-2-en-1-ol (prenol) using the processes described above and/or preparing prenal using the processes described above via isoprenal (3-methylbut-3-en-1-al) made from isoprenol; and
C) condensing the prenal with prenol to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

Step A can be performed as described above or by other methods known in the art, preferably via distillation at a temperature at which the hemiformal is cleaved to formaldehyde and isoprenol, so that the formaldehyde can be easily separated from the isoprenol, and more preferably by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.

Step B comprises oxidative dehydrogenation of prenol and/or isoprenol. The conversion of isoprenol with a catalytically active metal catalyst forms a reaction mixture of 3-methylbut-3-en-1-al and 3-methylbut-2-en-1-al. The former isomer may then isomerize under base catalysis to give the desired 3-methylbut-2-en-1-al.

Step C can be performed as described above, for example via steps a) to c).

The thus obtained citral is a useful intermediate for, e.g., menthol or linalool.

Menthol may be prepared from citral via a process comprising the steps of
catalytic hydrogenation of citral to obtain citronellal;
cyclization of citronellal to obtain isopulegol in the presence of an acidic catalyst and
catalytic hydrogenation of isopulegol to obtain menthol.

The overall reaction sequence is illustrated by the reaction scheme below.

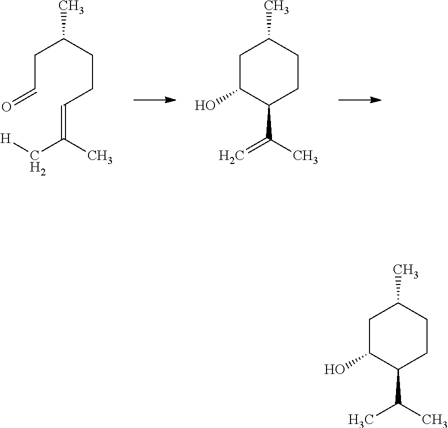

The hydrogenation of citral to obtain citronellal may be achieved by hydrogenation in the presence of a rhodium-phosphine catalyst.

The cyclization of citronellal to isopulegol may be achieved by cyclization in the presence of a Lewis-acidic aluminum-containing catalyst, such as a bis (diarylphenoxy) aluminum compound, which may be used in the presence of an auxiliary, such as a carboxylic anhydride. The isopulegol may be recovered from the catalyst-containing reaction product by distillative separation to give an isopulegol-enriched top product and an isopulegol-depleted bottom product. From the bottom product, the catalyst may be regenerated. The isopulegol obtainable in this way by the cyclization of citronellal can be further purified by suitable separating and/or purification methods, in particular by crystallization, and be at least largely freed from undesired impurities or by-products.

The hydrogenation of isopulegol may be achieved by hydrogenation in the presence of a heterogeneous nickel-containing catalyst, preferably a heterogeneous nickel- and copper-containing catalyst.

Further details regarding the reaction sequence from citral to menthol may be found in US 2013/46118 A1, which is incorporated by reference herein.

In one aspect, the invention thus relates to an improved process for the preparation of menthol by producing citral using the above processes and then producing menthol from the citral. Menthol may be prepared as described herein or by other methods known in the art.

Linalool may be prepared from citral via a process comprising catalytic hydrogenation of citral to obtain nerol and/or geraniol, and isomerization thereof.

The hydrogenation of citral to obtain nerol and/or geraniol may be achieved by hydrogenation in the presence of a supported ruthenium, rhodium, osmium, iridium or platinum catalyst, preferably a ruthenium catalyst supported on carbon black.

The isomerization of nerol and/or geraniol to obtain linalool may be achieved by isomerization in the presence of a tungsten catalyst, in particular a dioxotungsten (VI) complex. Further details regarding the isomerization of nerol and/or geraniol may be found in U.S. Pat. No. 7,126,033 B2.

In one aspect, the invention thus relates to an improved process for the preparation of linalool by producing citral using the above processes and then producing linalool from the citral. Linalool may be prepared as described herein or by other methods known in the art.

The present invention can be further explained and illustrated on the basis of the following figures and examples. However, it will be understood that these figures and examples are included merely for purposes of illustration and are not intended to limit the scope of the invention in any way.

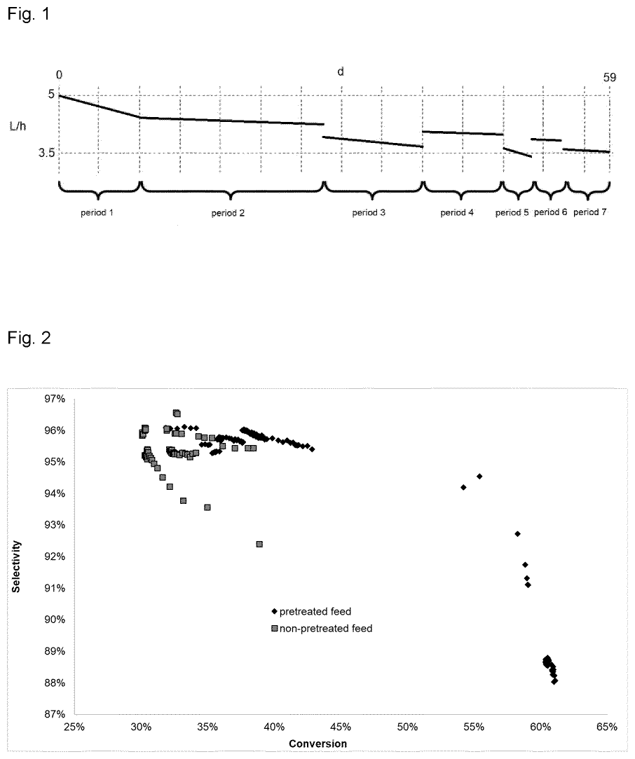

FIG. 1 shows the oxygen consumption in the liquid phase oxidation of prenol.

FIG. 2 shows the conversion and selectivity of the oxidation of prenol to prenal. Examples

### EXAMPLE 1—REMOVAL OF UROTROPIN FROM A PRENOL STREAM USING DIFFERENT ADSORBENTS

Different adsorbents are tested for their ability to remove urotropin from prenol. For this purpose, the relevant adsorbent is mixed with water at ambient temperature and stirred for 15 min for washing. Subsequently, the water-wet adsorbent is stirred with small amounts of the prenol (nitrogen-free) for 5 min. The prenol-wet adsorbent is then transferred to a column and the untreated prenol feed is run through the column (top down). The results are shown in table 1.

TABLE 1
Removal of urotropin from a prenol
stream using different adsorbents

| | | urotropin | urotropin |
|---|---|---|---|
| | | before adsorption | after adsorption |
| # | adsorbent | [ppm] | [ppm] |
| 1* | Lewatit CNP80 ^{[1]} | 35-51 | 11 |
| 2* | Purolite C104 | | 13 |
| | DL Plus ^{[2]} | | |
| 3* | Purolite C107 ^{[3]} | | 14 |
| 4* | BASF F-24X ^{[4]} | | 27 |
| 5* | Molecular sieve | | 5 |
| | BASF 4 Å ^{[5]} | | |
| 6* | Molecular sieve | | 17 |
| | BASF 13 X ^{[ ]} | | |
| 7* | BASF Selexsorb CDX ^{[6]} | | 3 |
| 8* | Chemviron Carbon | | 15 |
| | CPG LF ^{[7]} | | |
| 9* | Norit GAC 1240 ^{[7]} | | 7 |
| 10* | Chemviron Carbon | | 12 |
| | Aquacarb 207C ^{[8]} | | |
| 11 | Purolite S956 ^{[9]} | | 1 |
| 12 | Siral 40 ^{[10]} | | 0.5 |

^{[1]} Weakly acidic, macroporous cation exchanger based on acrylic
^{[2]} Polyacrylic porous weak acid cation ion exchange resin
^{[3]} Polyacrylic macroporous weak acid cation ion exchange resin
^{[4]} Acid active bentonite clay
^{[5]} Zeolithe (aluminosilicates mineral)
^{[6]} Alumina-based adsorbent
^{[7]} Activated carbon
^{[8]} Coconut Based Granular Activated Carbon
^{[9]} Macroporous crosslinked polymer with a phosphonic acid functionality
^{[10]} Weakly acidic silica-alumina, pore size 40 μm, high level of surface acidity
*comparative example

The examples show that Purolite S956 and Siral 40 show a significant behavior regarding the removal of urotropin (“organically bound nitrogen”) contained in the prenol stream (“ethylenically unsaturated alcohol stream”).

### EXAMPLE 2—REMOVAL OF UROTROPIN FROM A PRENOL STREAM USING SIRAL 40

A prenol stream having an initial content of 35 ppm of urotropin is passed through a column (15 mm inner diameter, 250 mm length) of 1 g of Siral 40 with a flow rate as indicated in table 2. Every 30 min, a sample was taken and the urotropin content of the treated prenol was determined by an oxidative combustion method with a chemiluminescence detector. The results are shown in table 2.

TABLE 2
Removal of urotropin from a prenol stream using Siral 40

| t [min] | prenol [mL/min] | urotropin [ppm] |
|---|---|---|
| 0 | 0 | 35 |
| 30 | 0.89 | 1 |
| 60 | 0.91 | 1 |
| 90 | 0.89 | 2 |
| 120 | 0.89 | 2 |
| 150 | 1.06 | 2 |
| 180 | 1.06 | 2 |
| 210 | 1.06 | 2 |
| 240 | 1.08 | 2 |
| 270 | 1.39 | 2 |
| 300 | 1.39 | 2 |
| 390 | 1.38 | 3 |

As can be seen from the results in table 2, the process of the present invention allows for significantly removing urotropin from prenol using Siral 40 as an adsorbent with no breakthrough during the duration of the test.

### EXAMPLE 3—OXYGEN CONSUMPTION IN THE LIQUID PHASE OXIDATION OF PRENOL

A reactor was charged with 103 g of a Pt/Al₂O₃ catalyst (0.9 wt.-% Pt). A feed stream containing 97 g/h of prenol and 7 g/h of water was metered through the reactor by using metering pumps. The prenol specifications (urotropin content) varied throughout the reaction periods 1 to 7 (see FIG. 1) as shown in table 3. The reaction was carried out for 59 d at an inlet pressure of 3.9 bara and an outlet pressure of 3.6 bara, and at an inlet temperature of 45° C. and an outlet temperature of 48° C. The oxygen consumption was determined quantitatively via the decrease of oxygen flow in L/h using a flowmeter. Oxygen consumption is a measure of catalyst activity.

TABLE 3
Prenol specifications throughout reaction periods 1 to 7

| | | urotropin content | |
|---|---|---|---|
| period | pre-treatment of prenol | of prenol [ppm] | |
| period 1* | — | 4 | ppm |
| period 2 | adsorption ^{[1]} using 1 wt.-% of Siral 40 | <2 | ppm |
| period 3* | — | 4 | ppm |
| period 4 | adsorption ^{[1]} using 0.5 wt.-% of Siral 40 | <2 | ppm |
| period 5 | adsorption ^{[1]} using 0.1 wt.-% of Siral 40 | 2 | ppm |
| period 6 | adsorption ^{[1]} using 0.5 wt.-% of Siral 40 | <2 | ppm |
| period 7 | adsorption ^{[1]} using 0.25 wt.-% of Siral 40 | <2 | ppm |

^{[1]} Carried out as shown in Example 2
*comparative example

The data of table 3 and FIG. 1 shows that the decline in oxygen consumption is less marked when using prenol streams pre-treated in an adsorption step according to the invention (periods 2 and 4 to 7) in comparison to non-pre-treated prenol streams (periods 1 and 3).

### EXAMPLE 4—SELECTIVITY TO PRENAL

A fixed-bed reactor containing a heterogeneous catalyst was filled with an unsaturated alcohol; i) treated with an adsorbent for removing nitrogen or ii) with an untreated feed containing up to 35 ppm nitrogen. In both cases, the reaction was run at 80° C. at 1.5 bar for several hours in order to compare the results regarding the catalyst activity and selectivity. The results are depicted in FIG. 2. It can be seen from FIG. 2 that the conversion to prenal was higher and the selectivity was higher when the organically bound nitrogen was removed from the prenol feed stream before the reaction, compared to the untreated prenol feed stream.

1.-18. (canceled)

19. A process for removing organically bound nitrogen from an ethylenically unsaturated alcohol stream, by contacting the alcohol stream with a weakly acidic solid adsorbent.

20. The process of claim 19, wherein the solid adsorbent is a crosslinked resin having phosphonic functional groups.

21. The process of claim 19, wherein the solid adsorbent is a silica-alumina hydrate.

22. The process of claim 19, wherein the ethylenically unsaturated alcohol stream is passed over a bed of the weakly acidic solid adsorbent.

23. The process of claim 19, wherein the ethylenically unsaturated alcohol stream comprises, after contacting the alcohol stream with a weakly acidic solid adsorbent, less than 2 ppm of organically bound nitrogen.

24. A process for the preparation of an ethylenically unsaturated aldehyde from an ethylenically unsaturated alcohol in the presence of an oxidant and a catalytically active metal catalyst, wherein prior to contacting with the catalytically active metal catalyst, the ethylenically unsaturated alcohol stream is treated by a process according to claim 19.

25. The process according to claim 24, wherein the catalytically active metal is selected from platinum, palladium and gold.

26. The process according to claim 24, wherein the catalytically active metal is deposited on a support.

27. The process according to claim 26, wherein the support is selected from carbonaceous materials and oxidic materials.

28. The process according to claim 24, wherein the oxidant is selected from oxygen and hydrogen peroxide.

29. The process according to claim 24, the process being carried out at a temperature in the range of from 1 to 250° C.

30. The process according to claim 19, wherein the reaction is performed in the presence of a liquid phase, which contains at least 25 wt.-% of water.

31. The process according to claim 30, wherein the liquid phase contains 1 to 75 wt.-% of the ethylenically unsaturated alcohol, based on the total amount of the liquid phase.

32. The process according to claim 24, wherein the ethylenically unsaturated alcohol is 3-methylbut-2-en-1-ol (prenol) and the ethylenically unsaturated aldehyde is 3-methylbut-2-en-1-al (prenal).

33. The process according to claim 32, wherein prenol is obtained by reacting a formaldehyde source and isobutylene to obtain 3-methylbut-3-en-1-ol (isoprenol), and subjecting at least part of the obtained isoprenol to isomerization.

34. A process for the preparation of 3,7-dimethyl-octa-2,6-dienal (citral) comprising:
obtaining prenal by the process according to claim 32:
condensing the prenal with prenol to obtain diprenyl acetal of prenal; and
subjecting the diprenyl acetal of prenal to cleaving conditions to obtain citral via prenyl (3-methyl-butadienyl) ether and 2,4,4-trimethyl-3-formyl-1,5-hexadiene.

35. A process for the preparation of menthol, comprising preparing citral by the process according to claim 34, and reacting citral to obtain menthol.

36. A process for the preparation of linalool, comprising preparing citral by the process according to claim 34, and reacting citral to obtain linalool.
